(19) United States
(12) Patent Application Publication
(10) Pub. No.: US 2010/0021990 A1
(43) Pub. Date: Jan. 28, 2010

(54) HIGH THROUGHPUT BIOPROCESS APPARATUS

(76) Inventors: Wade Edwards, Fish Hoek (ZA); Winston Daniel Leukes, Grassy Park (ZA); P.G. Lourens, Cape Town (ZA); Sheena Janet Fraser, Sea Point (ZA)

Correspondence Address: LAHIVE & COCKFIELD, LLP;FLOOR 30, SUITE 3000, ONE POST OFFICE SQUARE, BOSTON, MA, 02109, US

(21) Appl. No.: 12/296,888

(22) PCT Filed: Mar. 27, 2007
(86) PCT No.: PCT/IB2007/000764

§ 371 Date: Aug. 20, 2009

(30) Foreign Application Priority Data
Apr. 12, 2006 (ZA) 2006/02975

(51) Int. Cl. C12N 1/20; C12M 1/00

(52) U.S. Cl. 435/253.5; 435/289.1; 435/252.1

(57) ABSTRACT

The invention relates to a multiple bioreactor system comprising a plurality of bioreactors, a source of pressurised fluid, and distribution means for distributing the fluid to the bioreactors, wherein the bioreactor system includes backpressure creating means presented by, before or after each bioreactor and the source of pressurised fluid such that each backpressure creating means provides a resistance to the flow of the pressurised fluid which is greater than the resistance to flow between each backpressure creating means. The invention further relates to A method of operating a multiple bioreactor system comprising providing a plurality of bioreactors, a source of pressurised fluid, and distribution means for distributing the fluid to the bioreactors, wherein the bioreactor system includes backpressure creating means presented by each bioreactor or located between each bioreactor and the source of pressurised fluid such that each backpressure creating means provides a resistance to the flow of the pressurised fluid which is greater than the resistance to flow between each backpressure creating means and operating the system.

### BACKGROUND TO THE INVENTION

This invention relates to a multiple bioreactor system. In particular, this invention relates to a multiple bioreactor system using pressurized fluid.

In the biotech industry, most products are generated through some bioprocess involving a bioreactor. A considerable number of process parameters affect the outcomes and therefore the performance of a bioprocess. These include the nature of the production organism, the components and their concentrations and ratios of the growth and production medium, the pH and colligative properties of the growth medium, oxygen mass transfer, etc. In addition, a number of different bioreactor formats are available, e.g. Continually Stirred Tank Reactors (CSTRs), air-lift reactors and membrane bioreactors. Membrane bioreactors are very useful since they are continuous and allow changes of culture conditions over time to provide an optimum and inherently offer better performance in certain circumstances. Most process optimization is done empirically since it is currently not possible to accurately predict the optimal set of conditions from first principles. Thus many experiments are needed to find suitable and then optimal conditions for growth and product formation.

It would be preferable if these experiments could be done in parallel and/or sequentially without much turn-around time, as well as on a smaller scale to minimize materials used. Typically, multi-parallel studies in small scale systems like flasks or micro-titre plates are used, but they typically do not allow fed batch or continuous operation and are not scalable to production bioreactors. Membrane bioreactors simulate the natural environment of microbes by providing a solid/liquid (air) interface and have been shown to generate significant bio-process enhancements. Thus, small scale, multiple mini-reactors are very useful for rapid screening and optimization of conditions for the operation of lab to large scale units. Scale up is easy from the small to large scale units. Such bioreactors have been reported in literature, but these have been driven by multi-channel pumps. These pump drives have pulsatile and uneven flow for the liquid side and are expensive. The air flow distribution is normally kept constant by trial and error by adjusting back-pressure on each bioreactor/module.

Alternatively individual air supplies are necessary for each bioreactor/module, which becomes costly.

A need exists for a multiple bioreactor system which exhibits substantially identical conditions in each bioreactor driven by a source of pressurised fluid.

### SUMMARY OF THE INVENTION

According to a first aspect to the present invention there is provided a multiple bioreactor system comprising:
a plurality of bioreactors,
a source of pressurised fluid, and
distribution means for distributing the fluid to the bioreactors,
   wherein the bioreactor system includes backpressure creating means presented by, before or after each bioreactor and the source of pressurised fluid such that each backpressure creating means provides a resistance to the flow of the pressurised fluid which is greater than the resistance to flow between each backpressure creating means.

Preferably the bioreactors are located in parallel within the bioreactor system. The bioreactors are preferably membrane bioreactors, either single fibre membrane bioreactors of multi-fibre membrane bioreactors. Most preferably the bioreactors comprise at least one hollow fibre membrane, for example a capillary membrane, preferably enclosed in a shell.

In a preferred embodiment of the present invention the backpressure creating means are flow regulating valves, nozzles or frits, as in example 1. However, it will be appreciated that the bioreactor itself may present or be the backpressure creating means. Where the bioreactor is a membrane bioreactor, the membranes themselves may present the backpressure creating means, subject always to the fluid pressure resistance across the membrane being much greater than resistance between membranes, as in example 2.

In a preferred embodiment of the present invention, the fluid is a gas, most preferably air. However, it will be appreciated that the fluid may also be a liquid, for example a nutrient medium supplied to the lumen of the hollow fibre membranes. Nutrient medium may pass through the lumen of the hollow fibre membranes and a biofilm may grow on an outer surface of the hollow fibre membranes, sustained by the nutrient medium passing through the walls of the hollow fibre membranes. Biofilm permeate including excess nutrient medium and product of the biofilm can be recovered from the reactor. Product may be isolated from the permeate and so recovered. Nutrients may also be monitored to ascertain growth kinetics of the biofilm. In a most preferred embodiment to the present invention, the gas drives the supply of liquid nutrient to the bioreactors.

According to a second aspect to the present invention there is provided a method of operating a multiple bioreactor system comprising the steps of providing a plurality of bioreactors, a source of pressurised fluid, and distribution means for distributing the fluid to the bioreactors, wherein the bioreactor system includes backpressure creating means presented by each bioreactor or located between each bioreactor and the source of pressurised fluid such that each backpressure creating means provides a resistance to the flow of the pressurised fluid which is greater than the resistance to flow between each backpressure creating means and operating the system.

### DETAILED DESCRIPTION OF THE INVENTION

The system allows for the operation of a number of reactors in parallel under very similar air flow, air pressure and liquid pressure conditions. The advantage of this arrangement is that the system according to the present invention allows:
The ability to determine biological effects of a culture or the system under equivalent conditions across several bioreactors over time, i.e. to observe the changes that occur in parallel over many membranes over extended periods of time or sacrifice individual bioreactors for analysis to determine time course events.
The ability to optimize growth media in parallel, thereby significantly reducing process development time.
The ability to test different membranes for filtration efficiency and bio- and chemical compatibility.

According to the bioreactor of the present invention pressure and flow conditions can be changed to optimize process conditions relating to the performance of the culture, inter alia:
To compare a series of species or strains for the production of a certain compound under equivalent conditions in parallel; and/or
To produce a number of different products at small scale for example screening applications.

The system according to the present invention may typically comprise:
A single or multi-fibre bioreactor preferably of the type described in U.S. Pat. No. 5,945,002. The bioreactor is preferably small enough for limited use of space or materials.
A fluid (air) pressure source—typically an air compressor or gas cylinder.
A manifold distributing the pressurised fluid to a number of pressure vessels including a pressure vessel containing growth medium, for example a nutrient liquid, which vessel includes a cap allowing correct distribution of pressure and liquid flow. The cap may have three connections, allowing pressurised fluid in, growth medium out and new media or other additives in.
Each pressure vessel is attached to the bioreactor either to the lumen or Extra Capillary Space (ECS) in the case of capillary membranes, depending on the operational requirements.
The bioreactors preferably contain one or more membranes with essentially equivalent range of resistance depending on tolerable differences in flux. This ensures even flux through the different bioreactors or flux in inverse proportion to the resistance offered.
For the growth of aerobic cultures, the air pressure source such as compressed air is required to distribute air through the membrane reactors. This is typically the same air supply that drives the growth medium.
If humidification is required, a humidifier may be connected to the air supply, preferably with a sterile filter on the inlet side. This is to allow sterile operation without the need for a special air filter that allows humidified air to pass through.
The humidifier can be a pressure vessel that includes a cap adapted to allow dry air under pressure in and pressurized, humidified air out.
The fluid distribution means, for example an air line, is preferably manifolded so that air can be distributed through all of the bioreactors.
The air line may be connected to each membrane module extra-capillary space.
The air and product outlet of each membrane reactor may be connected to a permeate collection vessel.
The permeate collection vessel is preferably a pressure vessel, preferably including a cap which may have three connectors, one to direct waste air and product into the vessel, one to remove product as required and one to allow air out.
The air outlet of the permeate collection vessel is preferably connected to a backpressure creating device, e.g. a flow regulating valve or a nozzle or frit of a predetermined specification.
The nozzles are substantially equivalent thereby allowing even air flow between the bioreactors, or flow in proportion to the resistance of the nozzles.
The nozzle specification determines the ratio of air flow rate to pressure.
The lumen side of the membranes within the bioreactor preferably has a prime line connected to a priming vessel. This allows the lumen to be primed and medium to be changed.
The priming vessel may have a cap with two connectors, one to let medium in, another to let medium out.
The air line and liquid lines preferably have in-line sterilisable pressure gauges.

It will be appreciated that the present invention may be used for pervaporation application with suitable modifactions.

The invention will now be described with reference to the following figures in which:

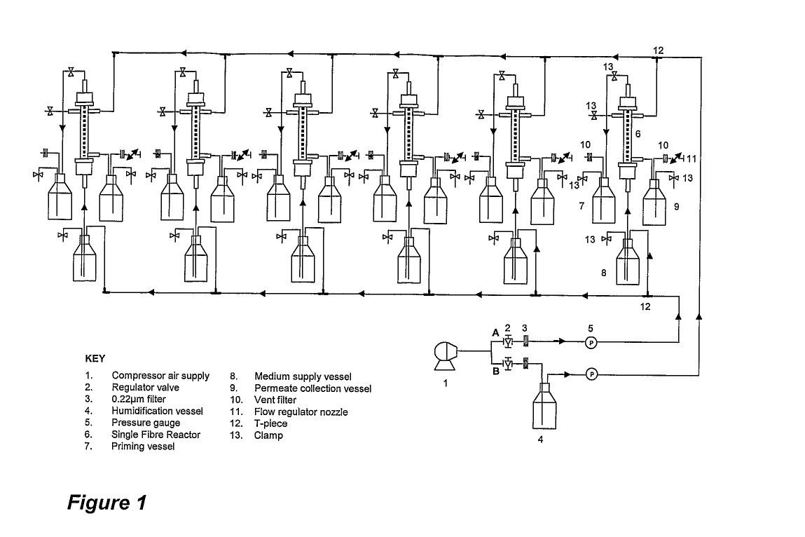

FIG. 1 is a schematic drawing of a multiple bioreactor system according to the invention.

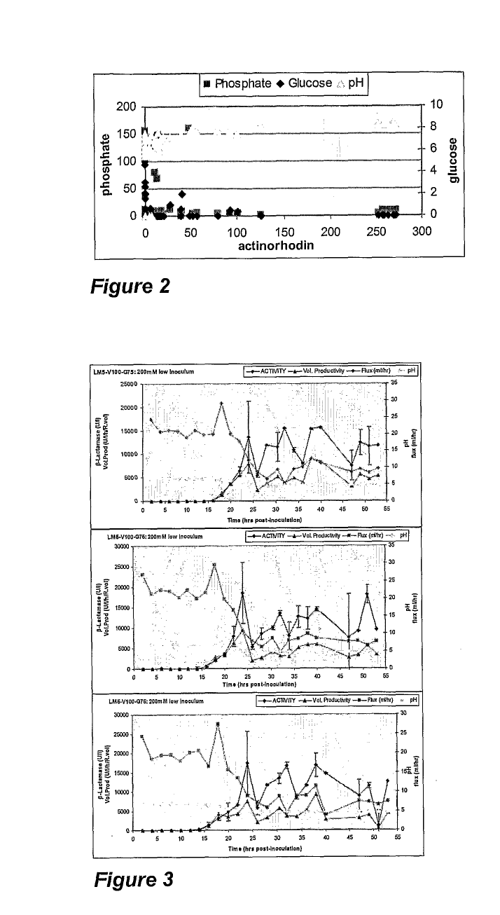

FIG. 2 is an XY graph showing relationship between pH, glucose and phosphate levels of permeate vs. actinorhodin production.

FIG. 3 shows time course-profiles for Single Fibre Reactors (SFRs) cultured using LM5-V100-G75 with 200 mM K—PO4 buffer, pH 7.2 and 1/50^{th} the inoculum concentration.

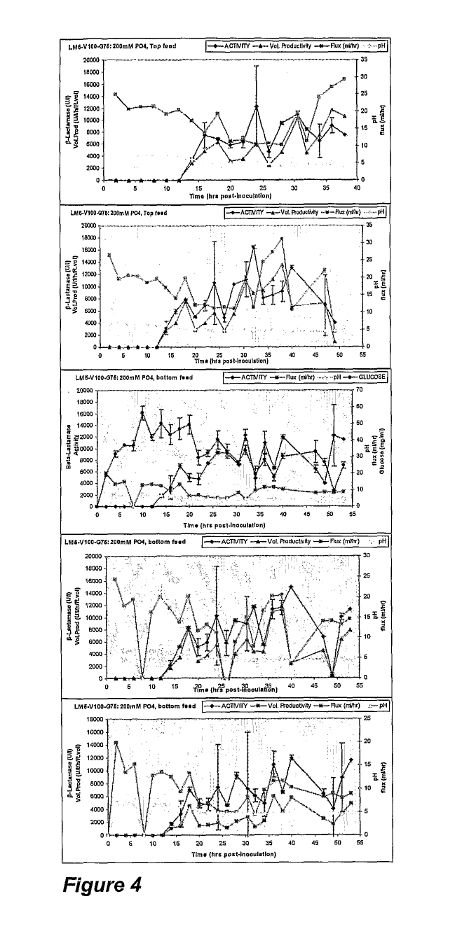

FIG. 4 shows time course-profiles for SFR's cultured using LM5-V100-G75 with 200 mM K—PO4 buffer, pH 7.2 cultured with 1× inoculum and fed with medium from either top or bottom manifold inlets.

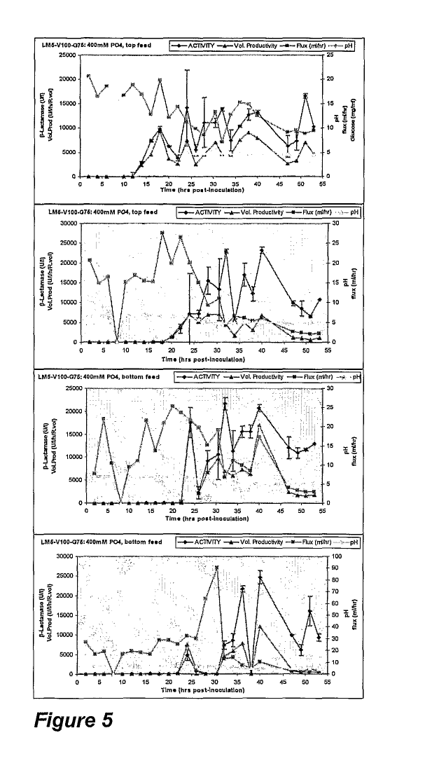

FIG. 5 shows time course-profiles for SFR's cultured using LM5-V100-75 with 400 mM K—PO4 buffer, pH 7.2 cultured with 1× inoculum and fed with medium from either top or bottom manifold inlets.

In FIG. 1 a compressor air supply 1 drives a bifurcated air line A, B, each line regulated by a regulator valve 2 followed by a 0.22 μm filter 3. Air line B enters a humidification vessel 4 and humidified air leaves the vessel through a pressure gauge 5 which is also located on line A.

Six single fibre bioreactors 6 are included in the system. Each bioreactor comprises a single membrane hollow fibre comprised of a capillary material, for example Al₂O₃(not shown). Air line A through six T-pieces 12 in series enters a medium supply vessel 8 for each bioreactor 6. Each vessel 8 includes a cap including an inlet for the airline A, an outlet for the medium and an inlet for changing or spiking of the nutrient content of growth medium which, in use, is clamped with a clamp 13. The pressure created within the vessel 8 on the surface of the medium by the inflowing air drives medium through the hollow fibre membrane, through an open clamp 13 and into a priming vessel 7 which, in use, is clamped off with a clamp 13. The priming vessel 7 has a cap including an inlet for the medium, a outlet clamped with a clamp 13 for emptying of the priming vessel when full, and an air outlet governed by a vent filter 10.

Airline B through a series of T-pieces located in series supplies air to the lumen of each bioreactor, i.e. to the outside of each hollow fibre. The air leaves the shell of the bioreactor through a vent which, in use, is clamped with a clamp 13 or through a second exit which drains to a product collection vessel 9. Medium which has flowed (permeated) through the hollow fibres, including product of a biofilm growing on an outer surface of each hollow fibre, and air passes into the vessel 9 which includes a cap including an inlet for the product, an outlet for draining the product bottle which, in use is clamped with a clamp 13 and a further vent for the air governed by a vent filter 10 and a flow regulator nozzle.

In use, both the supply of air and medium to each bioreactor is substantially equal because backpressure creating means creates a pressure from each bioreactor which is greater than the pressure between bioreactors. In so doing, flow rates which vary between bioreactor are limited in the operation of multiple bioreactors in parallel which allows for high throughput under similar conditions (useful in production) and/or process optimisation (useful in research and development operations).

It will be appreciated that the single fibre reactors illustrated above could be replaced by multi fibre reactors or indeed any other type of bioreactor requiring a supply of fluid(s). Pressure could be controlled either manually or automatically.

It will also be appreciated that either manual or automated control may be used to adjust or regulate the pressure and/or fluid supply to each reactor.

The invention will now be described with reference to the following non-limiting examples.

### Example 1

### Aerobic Mode

Optimisation of the Production of Actinorhodin by Streptomyces coelicolor A3(2).

In this example the backpressure creating means are nozzles positioned at the air outlet of each SFR.

The experiment was designed to assess the effects of nutrient feed rate, nutrient concentration and oxygenation on the production of actinorhodin by S. coelicolor. In addition, the influence of inoculum size on biofilm formation and productivity was also assessed. Altered process parameters were implemented consecutively or concurrently on each of 12 SFRs inoculated with S. coelicolor.

Actinorhodin levels are reported as total blue pigment, as quantified spectrophotometrically using SOP based on methods described by Ates et a/1997 (E1%, 1 cm=355).

### Sterilisation

SFR's were autoclaved and setup for aerobic operation according to standard operating procedures (SOPs). Autoclaved growth medium was dispensed into each of the medium supply vessels prior to starting the experiment.

### Inoculation

SFRs 1-5 were inoculated with 1 ml of spore suspension prepared from a single agar plate immersed with 10 ml sterile distilled water. SFRs 6-10 were inoculated with 1 ml 4 day flask culture incubated at 28° C. Inoculum was injected directly into the ECS of each SFR module using standard sterile technique. Immobilisation of inoculum on the outer surface of capillary membranes was completed according to SOPs.

### Operation

SFRs were operated under aerobic conditions according to SOPs. Initial pressures were set around 30 kPa. Medium supplied via line A from the lumen side of membrane conduits was manually set such that the pressure differential across the membrane surface from lumen to shell side was used to control the rate of nutrient feed (flux) to the biofilm. Permeate was collected and sampled daily from permeate collection vessels.

During optimisation of nutrient type and concentration current growth medium was either replaced with a fresh nutrient source by draining old growth medium into the prime bottle and refilling the medium supply vessel with the appropriate new medium type. Further, simple addition of nutrients or additives into initial growth medium was achieved by simply spiking remaining growth medium to give a known final concentration of the desired nutrient.

When evaluating the effect of increased oxygenation the compressed air was replaced with oxygen supplied using a technical grade oxygen cylinder.

TABLE 1
Culture conditions for each of 12 SFRs are tabulated below

| | Inoculum | Start- | | | | |
|---|---|---|---|---|---|---|
| SFR | type | up | Day 13 | Day 15 | Days 16-20 | Day 26 |
| 1 | mycelial | ISP2 | 30 kPa | O₂ | Spiked with | — |
| | | | | | glucose | |
| 2 | mycelial | ISP2 | 30 kPa | O₂ | Spiked with | — |
| | | | | | glucose | |
| 3 | mycelial | ISP2 | 30 kPa | O₂ | Spiked with | ISP2 |
| | | | | | glucose | |
| 4 | mycelial | ISP2 | 30 kPa | O₂ | Ates et al. 1997 | |
| | | | | | medium | |
| 5 | mycelial | ISP2 | 30 kPa | O₂ | Bystrykh et al. | ISP2 |
| | | | | | 1996 (Low PO₄ | |
| | | | | | medium) | |
| 6 | spores | ISP2 | Increased | Air | — | — |
| | | | to 60 kPa | | | |
| 7 | spores | ISP2 | Increased | Air | — | ISP2 |
| | | | to 60 kPa | | | |
| 8 | spores | ISP2 | Increased | Air | — | — |
| | | | to 60 kPa | | | |
| 9 | spores | ISP2 | Increased | Air | Ates et al. 1997 | ISP2 |
| | | | to 60 kPa | | medium | |
| 10 | spores | ISP2 | Increased | Air | Bystrykh et al. | — |
| | | | to 60 kPa | | 1996 (Low PO₄ | |
| | | | | | medium) | |

### Biofilm Development

Using either mycelial or spore inoculum biofilm growth was apparent within 24-48 hrs as S. coelicolor developed as small yellow coloured colonies along the membrane length. Colonies expanded, changing colour from yellow to orange-red in colour and became interconnected (72-120 hrs), forming a slightly tapering biofilm. Growth with International Streptomyces Project (ISP)₂ medium was rapid. As the biofilm began to differentiate the shiny orange-red colour turned opaque, white and then grey as differentiation and sporulation occurred (240-300 hrs). In all cases differentiation began on sections of membrane near the top of vertical SFRs. This appeared to be a function of medium flux. The appearance of a red pigment indicating actinorhodin in the medium coincided with sporulation. Differentiated biofilm turned blue-black, the pH of spent broth increased and more pigment was released with increased sporulation. Similarly with increased spent broth pH the pigmented medium turned from red to blue-purple due to the indicator characteristics of actinorhodin.

SFRs inoculated with spores (1-5) did not develop as rapidly or into as thick a biofilm as observed for SFRs inoculated with mycelium (6-10). While operated at the same DP reactors inoculated with spores showed lower flow rates due to the development of a more dense biofilm, facilitating a greater resistance to nutrient flow through the membrane/biofilm and into the ECS. Differences in the extent of differentiation and pigmentation within the same bank are the result of variability in nutrient supply to the developing biofilm (flux) caused by differences in membrane/biofilm resistance and/or reactor history. Under identical inoculation and/or culture conditions inherent differences in membrane resistance may be used to determine the robustness of a production process.

This may however have been influenced by slower flow rates even though similar ΔP was used for both banks of SFRs. Even within replicates differentiation and pigmentation showed differences that appeared to be dependent on flow rate and/or reactor history.

### Productivity

Actinorhodin concentrations and SFR volumetric productivity, calculated over a 360 hr period (from 14 days post-inoculation), are recorded in Table 2. On average, SFRs inoculated with mycelia showed more rapid biofilm formation and earlier onset of actinorhodin production, while those inoculated with spores and operated at 60 kPa under air showed greater overall actinorhodin production. Actinorhodin production was induced by exposing the biofilm to pure oxygen; however increased actinorhodin levels were not sustained. Of the 3 growth medium selected, ISP2 growth medium containing 4 g/l glucose was the most productive.

TABLE 2
Actinorhodin Production by different SFRs.

| | Actinorhodin | | | Volumetric Productivity | | |
|---|---|---|---|---|---|---|
| | (mg/l) | | | (mg/l/h/reactor volume) | | |
| SFR | Maximum | mean | SD | Maximum | mean | SD |
| 1 | 129.27 | 30.18 | 28.57 | 16.50 | 2.09 | 3.11 |
| 2 | 119.65 | 13.16 | 20.86 | 13.73 | 1.17 | 2.47 |
| 3 | 219.76 | 68.88 | 57.56 | 30.15 | 6.10 | 6.86 |
| 4 | 181.64 | 29.05 | 33.60 | 5.78 | 1.97 | 1.54 |
| 5 | 67.42 | 24.01 | 14.85 | 6.62 | 1.82 | 1.32 |
| 6 | 110.09 | 17.70 | 23.34 | 5.75 | 1.30 | 1.55 |
| 7 | 223.62 | 48.97 | 56.38 | 11.33 | 2.72 | 3.06 |
| 8 | 206.05 | 58.44 | 54.32 | 15.73 | 3.61 | 3.59 |
| 9 | 269.62 | 69.60 | 92.24 | 15.99 | 3.16 | 3.79 |
| 10 | 25.23 | 7.34 | 7.98 | 1.17 | 0.41 | 0.37 |

Kinetic analysis of SFRs showed a trend towards increased actinorhodin production at higher pH and lower glucose or phosphate levels (e.g. FIG. 2). This trend was confirmed by statistical analysis. However, these correlations were not significant (Table 3).

TABLE 3
Correlation of substrate utilization with actinorhodin production showing
Pearsons Correlation coefficients (+1 > r > −1) below.

| SFR | 1 | 2 | 3 | 4 | 5 | 6 | 7 | 8 | 9 | 10 |
|---|---|---|---|---|---|---|---|---|---|---|
| Actinorhodin | 0.543 | 0.364 | 0.829 | 0.411 | 0.538 | 0.491 | 0.517 | 0.657 | 0.429 | 0.402 |
| vs. pH | | | | | | | | | | |
| Actinorhodin | −0.251 | −0.065 | 0.095 | −0.304 | −0.347 | −0.442 | −0.392 | −0.447 | −0.281 | −0.270 |
| vs. Glucose | | | | | | | | | | |
| Actinorhodin | Nd | nd | −0.165 | nd | nd | nd | nd | nd | −0.243 | nd |
| vs. Phosphate | | | | | | | | | | |

### Example 2

### Anaerobic Mode

Optimisation of β-Lactamase Production in Lactococcus lactis.

In this example the backpressure creating means are the membranes themselves.

The experiment was designed to assess the effects of increased buffer concentration in growth medium as a means of stabilising pH and recombinant protein production in SFRs. In addition, the effect of inoculum size on biofilm formation and the influence of Top or Bottom medium feed configuration on nutrient supply and utilisation was assessed. β-lactamase activity was quantified spectrophotometrically using SOP based on the Nitrocefin method (Oxoid).

### Sterilisation:

SFR's were autoclaved and set up for anaerobic operation according to (SOPs). Filter sterilized medium was dispensed into each of the medium supply vessels prior to starting the experiment.

### Inoculation:

SFR's were each inoculated with 1 ml of either 1× or 1/50^{th} L. lactis PRA290 (β-lactamase) pre-inoculum, cultured in ‘M17-G5 growth medium at 30° C. for 16 hrs. Inoculum was injected directly into the ECS of each SFR according to SOPs. Following inoculation medium was supplied to each SFR at 8 kPa overnight.

### Operation:

SFR's were manifolded in banks of 6 SFR's. Each SFR was supplied with medium from its own supply vessel. Within each bank, replicate SFR's were supplied with either LM5-V100-G75 containing 200 mM or 400 mM K—PO4 buffer (pH 7.2) fed from medium inlets situated either at the top or bottom of the glass manifold. Flux, pH and β-lactamase activity were assessed on fresh samples. Glucose and Protein levels were monitored collectively.

For each bank medium supply was regulated using pressure control valves. SFRs were monitored every 2 hrs post-inoculation. pH profiles of permeate were used to monitor growth and were also used as a basis for the adjustment of flux. Pressures were adjusted as follows:

| Time post-inoculation (hrs) | Pressure (kPa) |
|---|---|
| 0 | 8 |
| 16 | 13 |
| 22 | 18 |
| 28 | 30 |
| 30 | 50 |
| 34 | 70 |
| 36 | 80 |

### Biofilm Development

50 hrs post-inoculation a dense biofilm of the consistency of thick yogurt was apparent for all SFRs. This biofilm appears to be formed by the retention of L. lactis cells in exponential growth, by the membrane under high pressure. As the biofilm increases, resistance to flow also increases. Towards the end of the experiment, at pressures approaching 100 kPa, flux was reduced below the critical point required for immobilisation, resulting in planktonic growth.

### Productivity

SFR's cultured using a lower inoculum size showed a delay in pH decline and β-lactamase production by 4-6 hrs (FIG. 2) in contrast to control SFRs inoculated (FIG. 3). Neither maximum enzyme activities nor production stability differed significantly between SFRs cultured with the different inocula.

Initial growth appeared to be inhibited by 400 mM K—PO4 buffered medium. In these SFRs onset of enzyme production varied from 12-22 hrs post-inoculation in replicates, being most pronounced in bottom fed SFR (FIGS. 3 and 4). However, under high buffer concentrations maximimum β-lactamase levels were recorded (20000-24000 U.L⁻¹).

References set out below are considered incorporated herein by reference.
1. Ates S., Elibol M. and Mavituna F. (1997) Production of actinorhodin by Streptomyces coelicolor in batch and fed-batch cultures; Process Biochem 32: 273-278.
2. Bystrykh L. V, Ferna'ndez-Moreno M. A, Herrema J. K, Malparida F., Hopwood D. A and Dijkhuizen L. (1996) Production of Actinorhodin-Related “Blue Pigments” by Streptomyces coelicolor A3(2); J. Bacteriol. 178: 2238-2248.

1. A multiple bioreactor system comprising:
a plurality of bioreactors,
a source of pressurized fluid,
distribution means for distributing the fluid to the plurality of bioreactors, and
a plurality of backpressure creating means presented by, before or after each of said plurality of bioreactors and the source of pressurized fluid for providing a resistance to the flow of the pressurized fluid which is greater than the resistance to flow between each backpressure creating means of said plurality of backpressure creating means.

2. The multiple bioreactor system as claimed in claim 1, wherein the plurality of bioreactors are disposed in parallel within the bioreactor system.

3. The multiple bioreactor system as claimed in claim 1, wherein said plurality of bioreactors are membrane bioreactors.

4. The multiple bioreactor system as claimed in claim 3, wherein each of said membrane bioreactors comprise a single fibre membrane bioreactor or multi-fibre membrane bioreactor.

5. The multiple bioreactor system as claimed in claim 1, wherein each of the plurality of bioreactors comprise at least one hollow fibre membrane.

6. The multiple bioreactor system as claimed in claim 5, wherein the hollow fibre membrane comprises a capillary membrane.

7. The multiple bioreactor system as claimed in claim 1, wherein each of said plurality of backpressure creating means comprises a flow regulating valve, a nozzle, or a frit.

8. The multiple bioreactor system as claimed in claim 1, wherein each of said plurality of backpressure creating means to forms part of the bioreactor.

9. The multiple bioreactor system as claimed in claim 3, wherein each of said plurality of backpressure creating means comprises the membrane.

10. The multiple bioreactor system as claimed in claim 1, wherein the source of pressurized fluid is a gas.

11. The multiple bioreactor system as claimed in claim 1, wherein the source of pressurized fluid is air.

12. The multiple bioreactor system as claimed in claim 1, wherein the source of pressurized fluid is a liquid.

13. The multiple bioreactor system as claimed in claim 12, wherein the liquid is a nutrient medium.

14. The multiple bioreactor system as claimed in claim 13, wherein each of said plurality of bioreactors comprises a hollow fibre membrane, and wherein the nutrient medium is supplied to a lumen of the hollow fibre membrane.

15. The multiple bioreactor system as claimed in claim 14, wherein the nutrient medium passes through the lumen of the hollow fibre membrane.

16. The multiple bioreactor system as claimed in claim 1, wherein a gas drives a supply of liquid nutrients to one or more of said plurality of bioreactors.

17. The multiple bioreactor system as claimed in claim 5, wherein a biofilm grows on an outer surface of the hollow fibre membrane, the biofilm sustained by a nutrient medium passing through a wall of the hollow fibre membrane.

18. The multiple bioreactor system as claimed in claim 17, wherein biofilm permeate including excess nutrient medium and a product of the biofilm is recovered from the bioreactors.

19. A method of operating a multiple bioreactor system comprising
providing a plurality of bioreactors, a source of pressurized fluid, and distribution means for distributing the fluid to the bioreactors,
providing backpressure creating means presented by each bioreactor or located between each bioreactor and the source of pressurized fluid such that each backpressure creating means provides a resistance to the flow of the pressurized fluid which is greater than the resistance to flow between each backpressure creating means, and
operating the system.

20. (canceled)
